(19) United States
(12) Patent Application Publication
(10) Pub. No.: US 2020/0210872 A1
(43) Pub. Date: Jul. 2, 2020

(54) SYSTEM AND METHOD FOR DETERMINING COSMETIC OUTCOME EVALUATION

(71) Applicant: Steven H Dayan, Chicago, IL (US)

(72) Inventor: Steven H Dayan, Chicago, IL (US)

(21) Appl. No.: 16/732,158

(22) Filed: Dec. 31, 2019

Related U.S. Application Data
(60) Provisional application No. 62/787,166, filed on Dec. 31, 2018.

(51) Int. Cl. G06N 7/00; G06F 16/26; G06N 5/02; G16H 30/40; G16H 70/20; G16H 50/70

(52) U.S. Cl. CPC G06N 7/005; G06F 16/26; G16H 50/70; G16H 30/40; G16H 70/20; G06N 5/027

(57) ABSTRACT

A method, system and computer readable medium provides for a multi-dimensional data integration for estimating cosmetic outcomes in a computing environment including collecting sample data associated with cosmetic outcomes; receiving input defining one or more schemas for organizing the collected sample data; profiling the sample data to determine one or more metrics associated with the sample data, the metrics including at least a physical beauty metric, a genuineness metric and a self-esteem metric; generating one or more rules based on the one or more schemas, the rules including at least an attractiveness rule; and generating a functional estimation system based on the generated one or more rules, for use in processing a data input, the generated functional type system providing an estimation of cosmetic outcome.

### CROSS-REFERENCE TO RELATED APPLICATIONS

The present application is a non-provisional filing of, and claims benefit under 35 U.S.C. § 119(e) from, U.S. Provisional Patent Application Ser. No. 62/787,166, filed Dec. 31, 2018, entitled “SYSTEM AND METHOD FOR DETERMINING COSMETIC OUTCOME EVALUATION”, which is incorporated herein by reference in its entirety.

### BACKGROUND

A meteoric expansion in esthetic medicine followed the introduction of nonsurgical cosmetic neuromodulators and fillers in the early 2000s, which has been recently declining. The initial inertia has likely been tempered by the cosmetically interested patient's fear of appearing unnatural. Defining a natural outcome is a mission essential to the advancement of esthetic medicine; however, achieving a natural result following cosmetic intervention is a misnomer and a logically flawed endeavor that cannot exist. The ever elusive “natural” that is loudly desired by the populous, and ardently pursued by both industry and physicians, can only be an interpreted deduction.

Is esthetic medicine entering a period of darkness repressed by a clergy and congregation beholden to a liturgy that describes an unobtainable paradise? Until we enlighten ourselves beyond the cannons preaching beauty as a singular goal promising salvation, we may be doomed to a period of arrested development. Similar to the pre-enlightenment period where the prevailing thought was that the world was flat, today's conventional thinking decrees beauty, a one dimensional entity, at its center of faith. Beauty has become a highly simplified measurement for defining a pleasing outcome in esthetics and is an incomplete representation of what a human race, blessed with a consciousness, wants, and seeks in self and others. Large lips, inflated cheeks, projected chest, and behinds is a distortion of an evolutionary purified beauty. A generation that chooses to worship these cartoon-like ideals and its leaders cannot help but to also feel an inexplicable unease. When the priestess of reality TV with arguably the most perfectly balanced and mathematically proportioned face is seen crying with tears running down her cheeks yet no movement in her facial musculature, a conflict arises in the mind of the perplexed. She is beautiful but not genuine. An esthetic medical establishment that cannot discipline itself from the shallow glamor of adulterated beauty is complicit in its dissemination. Instead of leading, esthetic medicine assembly caters to the primal urges of the vulnerable and naive, thereby justifying the misguided creation of humans with inanimate beauty. We have entered a postmodern era where we have the tools, skills, and knowledge to deliver a beauty that is beyond reality, but inadvertently, we are also delivering precisely the opposite of what the populous desires: an inauthentic caricature of beauty. It is for this reason that esthetic medicine has stalled. The Big Bang of nonsurgical cosmetic treatments was followed by an accelerating expansion, 679% growth from 1997 to 2015. The increase, however, has fallen to a relative 6.7% over the last year. We have only ourselves to blame when cries of wanting to look natural are heard but not heeded. By crowning a one-dimensional objective as our only goal, we stand guilty as charged.

A doctor consulted with a 45-year old woman who presented with a conundrum. “Doctor, I didn't wear any make up and let my toxin and fillers wear off so I would look natural and you could see me. I am interested in looking better, I am open to receiving more fillers, toxin, or even surgery. I'll do whatever you think is best but I want to look natural. What can you do for me?”

Her riddle, could not be answered. But it forced the doctor to contemplate, what is natural?

We are born natural as are those on the cover of National Geo-graphic magazine are natural, with large noses, loose jowls, and hanging necks. Is this the natural that our patients seek? In esthetic medicine, attempting to create a natural outcome does not and cannot exist. It is an unattainable goal based on a flawed logic. A “natural” appearance can only exist in the absence of esthetic interventions. Yet many have now set sail on a quest to find the ever-elusive natural outcome, but can it only be appreciated as an interpretation? Patients no doubt seek to be beautiful but they also want to simultaneously appear genuine or trustworthy. To achieve a natural outcome, starts by reaching the intersecting longitude and latitude planes of beautiful and genuine. These two primitively encoded navigational tools are not yet in themselves enough to direct us to the desired destination. The most desirable people are not solely the most beautiful or the most genuine.

What esthetic medicine voyagers seek is not just being beautiful and genuine in appearance but more importantly, they want to be attractive. Being attractive is a multidimensional nebula far beyond the singularity of physical beauty. The attractive embody confidence and capture the disinterested interest of others. We all know of a physically beautiful person, blessed with ideal features, yet they project an unattractive image. They walk into a room with their head down, an angry scowl or pompous lift projecting a negative aura. In contrast, those who enter with a confident swagger, a hinted smile, and a suggested approachability may not have the most mathematically perfect features but they are tantalizingly and overwhelmingly captivating. They ooze attractiveness, a kinetic, and complex primordial recipe that esthetic providers are attempting to define and deliver.

### SUMMARY

To better define a natural outcome, a novel model based on the theory of relativity is suggested. Attractiveness consists of a 3-dimensional area defined by beauty, genuineness, and self-esteem. When all three variables are at a maximum, a desirable attractive appearance is achieved that can be interpreted as “natural.” Similar to the time-space dilemma, attractiveness is relative, dynamic, and highly dependent on the position of the projector and the interpreter. The 3D cube of attractiveness is therefore contained within a 4th dimension that takes into account the perspective of the judger. Implementing what defines a natural outcome, esthetic medicine will be prepared to meet the needs of future generations.

A method for multi-dimensional data integration in a computing environment includes collecting sample data associated with cosmetic outcomes; receiving input defining one or more schemas for organizing the collected sample data; profiling the sample data to determine one or more metrics associated with the sample data, the metrics including at least a physical beauty metric, a genuineness metric and a self-esteem metric; generating one or more rules based on the one or more schemas, the rules including at least an attractiveness rule; and generating a functional estimation system based on the generated one or more rules, for use in processing a data input, the generated functional type system providing an estimation of cosmetic outcome.

In one or more embodiments, the one or more rules includes data rules based on the one or more schemas that are defined in terms of profiled data from a plurality of patients with known cosmetic outcomes; and relationship rules that define associations between patients and one or more judging observers.

In one or more embodiments, the one or more schemas operate as a reference ontology, for use in comparing, classifying, and evaluating a metadata schema for generating three-dimensional volumes defining attractiveness based on the one or more metrics.

In one or more embodiments, the reference ontology provides a variable and dynamic reference; and wherein the collecting sample data associated with cosmetic outcomes includes collecting a judging observer as a metric of cosmetic outcome.

In one or more embodiments, the method is performed in a cloud or cloud-based computing environment.

In one or more embodiments, the functional estimation system includes a user interface for interacting with a user to enable determining an estimation of outcome of cosmetic intervention including one or more of makeup, hair care, neurotoxins, fillers, surgery and hormonal replacement.

In one or more embodiments, the functional estimation system comprises an estimation system for determining an estimation of outcome of cosmetic intervention including one or more of makeup, hair care, neurotoxins, fillers, surgery and hormonal replacement.

Another embodiment is directed to a system for ontology analysis of a schema definition for use with a data integration or other computing environment, including one or more processors operable to: collecting sample data associated with cosmetic outcomes; receiving input defining one or more schemas for organizing the collected sample data; profiling the sample data to determine one or more metrics associated with the sample data, the metrics including at least a physical beauty metric, a genuineness metric and a self-esteem metric; generating one or more rules based on the one or more schemas, the rules including at least an attractiveness rule; and generating a functional estimation system based on the generated one or more rules, for use in processing a data input, the generated functional type system providing an estimation of cosmetic outcome.

In one or more embodiments, the one or more rules includes data rules based on the one or more schemas that are defined in terms of profiled data from a plurality of patients with known cosmetic outcomes; and relationship rules that define associations between patients and surgeons.

In one or more embodiments, the one or more schemas operate as a reference ontology, for use in comparing, classifying, and evaluating a metadata schema for generating three-dimensional volumes defining attractiveness based on the one or more metrics. In one or more embodiments, the reference ontology provides a variable and dynamic reference; and wherein the collecting sample data associated with cosmetic outcomes includes collecting a judging observer as a metric of cosmetic outcome.

In one or more embodiments, the system is performed in a cloud or cloud-based computing environment.

In one or more embodiments, the functional estimation system includes a user interface for interacting with a user to enable determining an estimation of outcome of cosmetic intervention including one or more of makeup, hair care, neurotoxins, fillers, surgery and hormonal replacement.

In one or more embodiments, the functional estimation system comprises an estimation system for determining an estimation of outcome of cosmetic intervention including one or more of makeup, hair care, neurotoxins, fillers, surgery and hormonal replacement.

Another embodiment is directed to a non-transitory computer readable storage medium, including instructions stored thereon which when read and executed by one or more computers cause the one or more computers to perform a method comprising: collecting sample data associated with cosmetic outcomes; receiving input defining one or more schemas for organizing the collected sample data; profiling the sample data to determine one or more metrics associated with the sample data, the metrics including at least a physical beauty metric, a genuineness metric and a self-esteem metric; generating one or more rules based on the one or more schemas, the rules including at least an attractiveness rule; and generating a functional estimation system based on the generated one or more rules, for use in processing a data input, the generated functional type system providing an estimation of cosmetic outcome.

In one or more embodiments, the functional estimation system includes a user interface for interacting with a user to enable determining an estimation of outcome of cosmetic intervention including one or more of makeup, hair care, neurotoxins, fillers, surgery and hormonal replacement.

In one or more embodiments, the functional estimation system comprises an estimation system for determining an estimation of outcome of cosmetic intervention including one or more of makeup, hair care, neurotoxins, fillers, surgery and hormonal replacement.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a graph illustrating embodiments of the present invention.

FIG. 2 is a three dimensional graph illustrating an embodiment of the present invention.

FIG. 3A is a three-dimensional graph illustrating an embodiment of the present invention.

FIG. 3B is a three-dimensional graph illustrating an embodiment of the present invention.

FIG. 3C is a three-dimensional graph illustrating an embodiment of the present invention.

FIG. 4 is several three-dimensional graphs illustrating an embodiment of the present invention.

FIG. 5 is a network environment for illustrating embodiments of the present invention.

FIG. 6 depicts a display and a user interface in accordance with some embodiments.

FIG. 7 depicts a system in accordance with some embodiments.

FIG. 8 depicts a method in accordance with some embodiments.

FIG. 9 depicts a second method in accordance with some embodiments.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Being attractive is relative to the observer and the projector. It is defined where the unidimensional forces of genuineness, beauty, and self-esteem intersect, resulting in a three-dimensional cube of attractiveness. Similar to the theory of relativity, which requires observers to view the time-space continuum from a perspective beyond themselves, a fourth dimension is necessary to appreciate the esthetic ideal of “natural.”

Embodiments herein provide for systems and methods that apply a Special Theory of Relativity for Attractiveness for defining the ideal esthetic in human appearance.

It is first necessary to define the variables:

Beauty: A form of subconscious, primal communication consistent, and universally employed throughout all of nature, relevant to all living species indicating a being's current health, fertility, and genetic value. Beauty is measured mathematically using the phi formula for facial features in static 2-dimensional photographs.

Genuineness: A form of subconscious communication relative to an observer that is indicative of the projector's trustworthiness and is measured dynamically during animation using a 7-point Likert scale by the observer.

Self-esteem: A dynamic, minimally mutable measure of one's perceived self-worth or value. Self-esteem is measured by a self-rated Self-Esteem scale.

Attractiveness: A consciously influenced and perceived dynamic form of communication that captures the interest of an observer. Attractiveness is affected by the prime variables of beauty, genuineness, and self-esteem and consists of the area within the three dimensions defined by these indices. Attractiveness is also influenced by expression, hair style, posture, culturally specific adornments, costume, cosmetics, and is relatively appreciated. Attractiveness is measured by a judger's (observer's) perceived first impression projected by the object in photographs but is better determined via dynamic video.

### Beauty

Defining beauty has been debated for millenniums. Although there are philosophical, cultural, religious, artistic, and mathematical viewpoints to consider, for our purposes, beauty serves as a primal form of communication indicating the current health and fertility status of a living being, along with the adaptive value of its genes. In such a model, beauty may be mathematically defined by measuring symmetry, body size ratios, and facial proportions. The most recognized mathematical equation for defining ideal beauty is the phi formula, which takes in to account the golden ratio of proportions. This divine formula encases the Vitruvian man as well as defines the gospels of esthetic surgery.

Humans have overcome the intrinsic laws of nature via the ability to modify one's mathematically defined and genetically programmed beauty. We can alter facial proportions, body ratios, change eye color and augment secondary sexual characteristics, tricking the naive into believing an appearance of health, youthfulness, and fertility that is only but a false representation. Just as human kind cannot overcome the speed of light constant without consequences, there is a limitation in overtaking the laws of beauty after which human nature may collapse. While a world of human Ken and Barbie Dolls may fulfill the mathematics dictates of beauty, they also have surpassed the point on the curve where they appear fertile, healthy, and attractive to observers other than themselves. Beauty exhibits a Gaussian distribution. As we increase cosmetic interventions from makeup to hair care, neurotoxins to fillers, surgery to hormonal replacement, we can approach the mathematical proportions of an ideal beauty, but once past a distinct point, additional cosmetic intervention leads to an opposite effect. Cosmetically overtreated individuals project an image of being less healthy and evolutionarily fit. The nadir or high point of the natural curve is where the planes of genuineness and beauty intersect

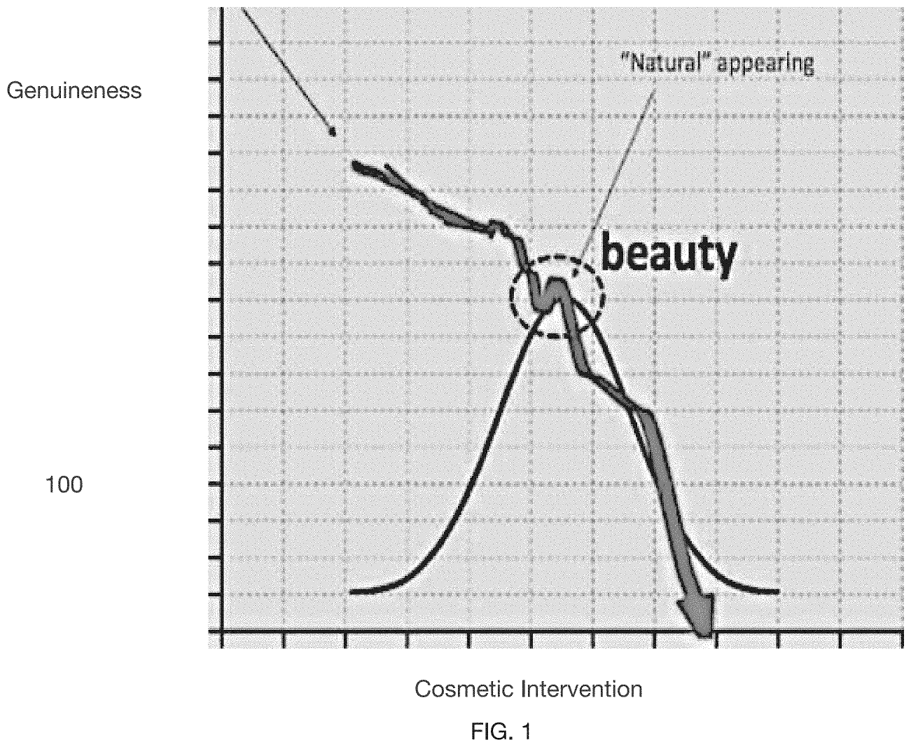

FIG. 1 illustrates a graph 100 comparing genuineness as a Y axis with cosmetic intervention as an X axis. As shown, genuineness starts out the highest at birth and can only be reduced with cosmetic intervention. The maximum point at which genuineness and beauty intersect is necessary for achieving a “natural” appearing result following cosmetic intervention.

Embodiments disclosed herein relate to systems and methods for identifying a natural appearing result following cosmetic intervention.

### Genuineness

Finding an ideal mate to procreate and promulgating genes is life's biological purpose from a Darwinian perspective, the most primal instinct of survival is to protect one's own genes. This aim is achieved by engaging with those who are believed to be genuine, whose actions and behaviors can be trusted. Genuineness is a sign of trustworthiness. Humans have developed an incredibly sensitive evolutionary preserved detector of genuineness. It is innately wired and housed deep within the subconscious brain.

Guillaume-Benjamin Duchene in 1862 describing a genuine smile wrote that while the zygomaticus major muscle was under conscious control and “follows the will,” the subconsciously controlled orbicularis oculi muscles are “put into play by the sweet emotions of the soul.” A genuine or Duchene smile sees both of these muscles contracting nearly simultaneously. Those who do not smile with their eyes may appear ingenuine or untrustworthy. Genuineness is a dynamically interpreted trait that requires a person to be viewed in animation. Cosmetic providers, however, have been marketed to use the amount of toxins or fillers that produce a significant improvement on two-dimensional static photos, likely blunting the appearance of a genuine Duchene's smile.

We are creating, validating, and teaching outcomes that result in cosmetically treated patients appearing ingenuine. It is not surprising patients are demanding to look natural. Appearing genuine is an essential component to cosmetic patients' desires. Those who are not trusted are generally not liked. In essence, trusted and genuine appearing people are more attractive. Rarely, if ever, however, does a cosmetic patient request to look more genuine. Furthermore, if genuineness was the goal, patients would never seek out cosmetic treatments as any intervention can only serve to reduce genuineness.

Creating beauty while protecting genuineness is at the core of what cosmetic patients desire, and this in itself can likely be used to define the “natural” results that patients seek, but this two-dimensional model attempting to define an esthetic ideal is not complete. Much like Newton's laws of motion breaks down at cosmic levels, we need to consider another dimension to find the ideal esthetic goal.

### Self-Esteem/Confidence

A person may be beautiful and genuine but not attractive. All cosmetic providers can recount a person who had a great outcome following cosmetic surgery or a nonsurgical treatment yet still does not capture the disinterested interest of another. They walk into the office with their head down, slumped shoulders, a shuffling gate, and hair draping over their face. One of the key components to being attractive is healthy self-esteem. The most engaging person in the room is often not the one with the most perfect facial features, but rather the one with the most confidence. Esthetic treatment can have a positive impact on self-esteem, confidence and happiness, and one's perception of their own appearance is highly correlated with their self-esteem.

### Attractiveness

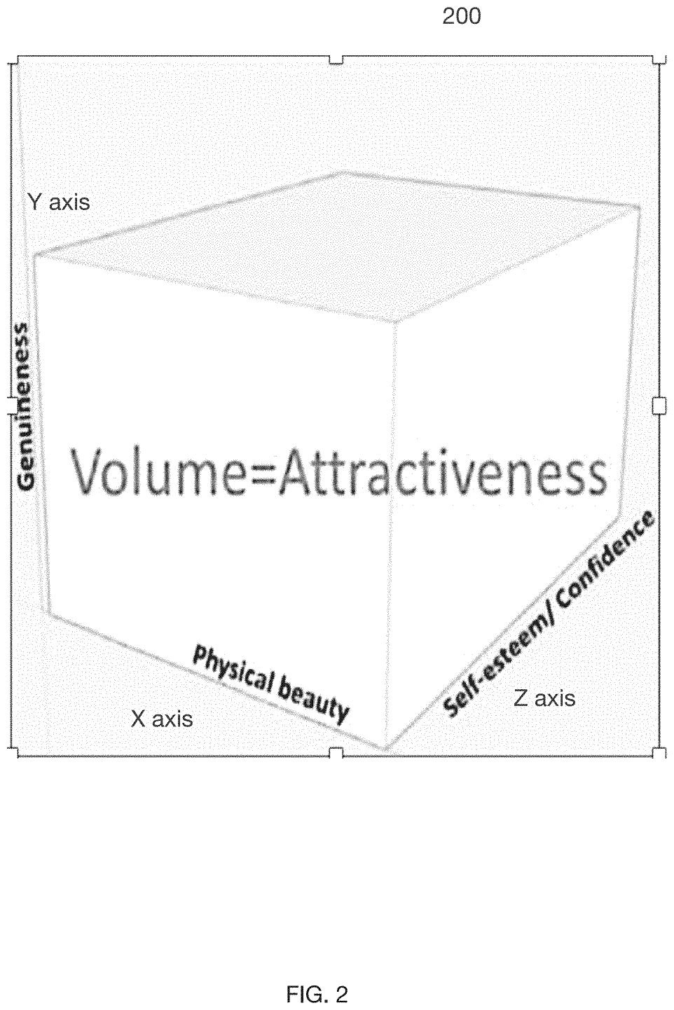

Referring to FIG. 2, graph 200 provides a three-dimensional view showing a Y axis of genuineness, an X axis of physical beauty and a Z axis of self-esteem/confidence.

Being attractive is what cosmetically seeking patient's desire and esthetic physicians aim to offer. The volume of the cube created by the three indices' of beauty, genuineness, and self-esteem defines attractiveness. As we increase cosmetic interventions, beauty can be increased as well as self-esteem. Genuineness, however, cannot be increased as humans are born genuine and it can only be decreased by cosmetic intervention. If judicious use of cosmetic intervention is employed, self-esteem and beauty can be increased while genuineness is preserved. It is at the nadir of these three parameters that the interpreted area of naturalness exists.

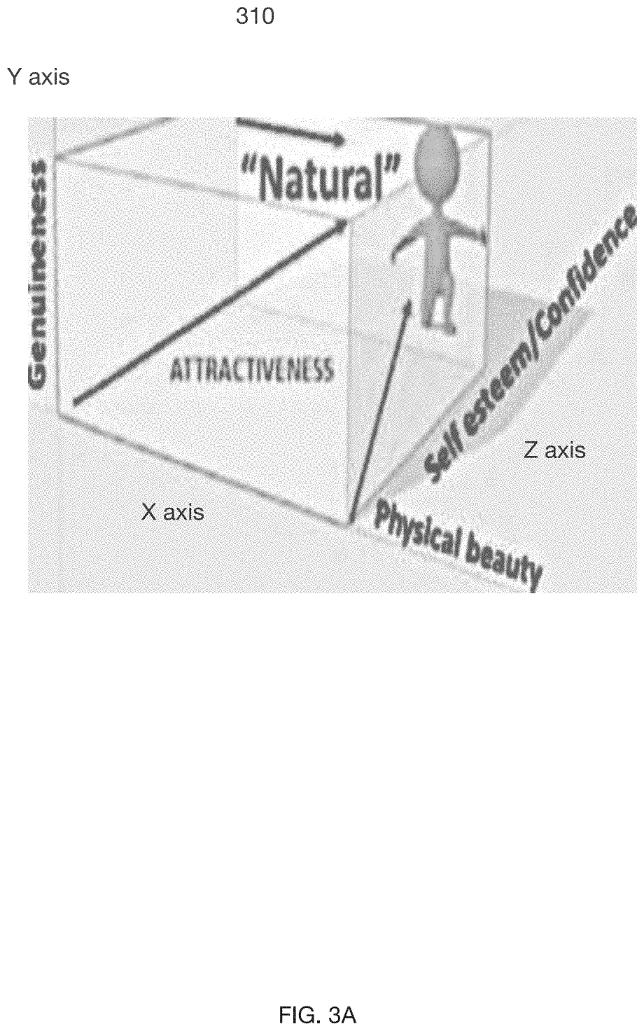

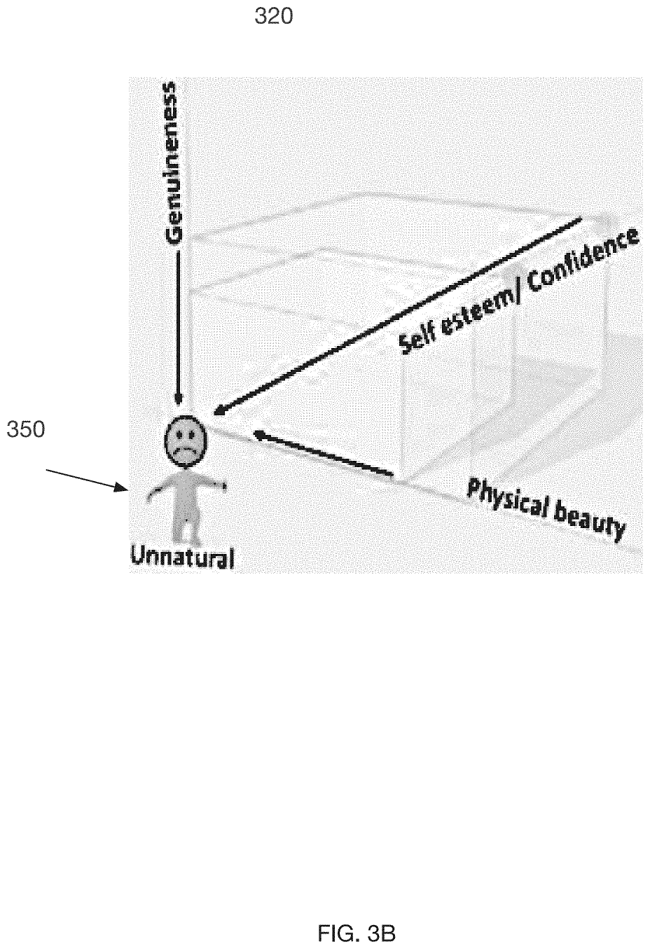

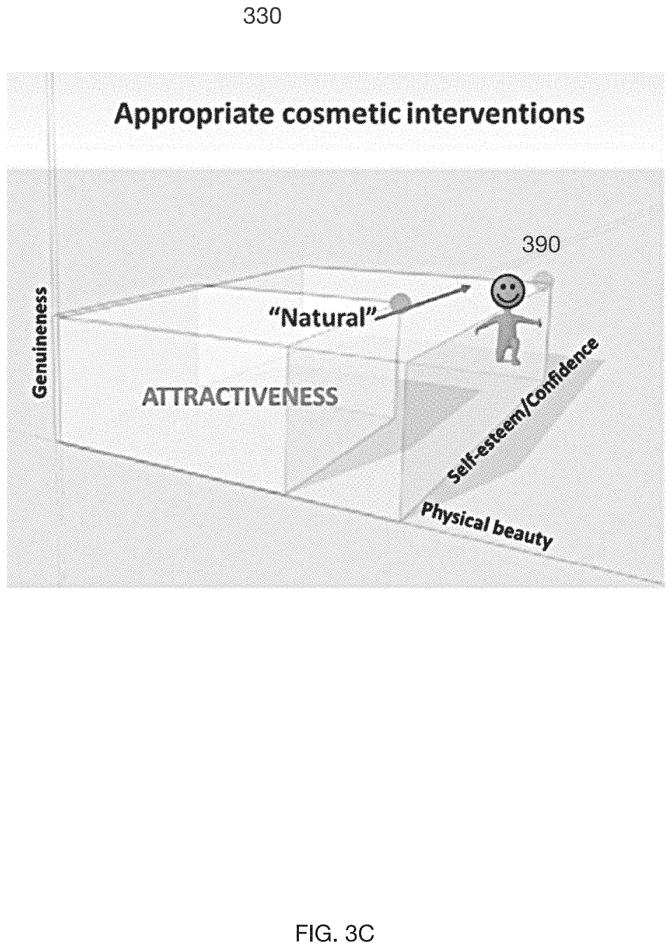

Referring to FIGS. 3A and 3B, the same three-dimensional graph shown as graph 310 and 320, respectively, illustrates how when cosmetic interventions are exaggerated so that ideal physical ratios defining beauty are surpassed, genuineness is reduced and self-esteem may drop as positive reinforcement from one's self and others wanes. Individual 350 is shown with an appearance that progresses outside the cube of attractiveness, the cosmetically treated person begins to assume the unnatural appearance that is feared.

Referring now to FIG. 3C, the same three-dimensional graph illustrates individual 390 as having appropriate cosmetic intervention. Naturalness can be interpreted as an area in the cube of attractiveness where beauty, genuineness self-esteem are at their maximum. Following appropriate cosmetic intervention, the cube of attractiveness may increase and naturalness may be preserved. If too much cosmetic intervention is employed then attractiveness can decrease and the individual may fall outside the cube of attractiveness and assume an unnatural appearance.

Although projected attractiveness may be increased or decreased via consciously controlled interventions such as costume, adornments, posture, expression, makeup, hair styles, and cosmetic medicine, it is most closely linked with an individual's self-esteem. A positive 59%-85% correlation between global self-esteem and self-rated attractiveness has been reported. When self-esteem is high, so is one's self-perceived and projected attraction level. This is not to be confused with narcissism which is an iteration of low self-esteem and is an unattractive character trait.

### Relativity of Attractiveness

Congruent with the time-space model where both time and space are dynamic and not absolutes relative to the observer, attractiveness is variable and relevant to the interrelatedness in space of the projector and the observer. Multiple studies have shown that ratings of attractiveness are influenced by perceptions of one's own attractiveness level. In a frequently cited study, participants rated their mate's level of attractiveness as 8.06/10 regardless of where either one of them fell on an objective scale of attractiveness. Accordingly, two attractive people will rate each other 8.06, as do two less attractive people who will also rate each other 8.06, but all four people would not score an 8.06 if rated by a large group of independent raters. Attractiveness, not beauty, is in the eye of the beholder. The embodiments described herein conclude that mates do not settle for an unattractive romantic partner, rather they seek out and find another who they believe to be attractive, and quite often their selected mate's objective attractiveness level is similar to their own. If circumstances change and self-esteem rises or falls, it may alter what one finds attractive. Attractiveness of another is relative to the perceiver's own impression of themselves. Clinically, this may be inferred that following a cosmetic treatment whether or not a patient feels satisfied with the result may be highly correlated with their self-esteem.

In addition, whether or not their acquaintances, family, and friends feel the result successfully increased the patient's attractiveness level is relative to that specific acquaintance, family member or friend's own self-esteem/attractiveness rating. Therefore, when treating a patient, taking into consideration the attractiveness level of those around them would be prudent, although admittedly impractical. The perceiver has to be open and willing to receive the impression to evaluate it, otherwise, the projector may not be recognized as attractive. To illustrate this point, in a celebrated social experiment reported by the Washington Post in 2007, Josh Bell, the famed violinist, played classical Bach on a Stradivarius violin in the Washington subway on a snowy cold January morning.

Few stopped or were attracted to the beautiful music. The vast majority passed by “other than a 3 year old.” Two days earlier, Josh played to a sold-out crowd in a Boston theater. What was the difference? Same guy, same violin, same music. It came down to the relative perception of the commuter who did not recognize and were not attracted to the beautiful music being projected. Similarly, if a person is sulking after having a bad day because of recently losing their job, their confidence and self-esteem levels may be low and even if a mathematically ideal, objectively beautiful, person walks past them they may not perceive them at all. If, however, in a different environment, a month later, these same two people are on vacation and the person who previously was disengaged now has a new job and is feeling confident, they may be more inclined to recognize, appreciate, and be attracted to that mathematically ideal person walking past them. Although beauty can existentially exist regardless of the projector or receiver, attraction requires at least two people and a consciously cognitive deduction. In a concept that has not been clearly defined before in esthetic medicine, projecting and being perceived attractive is a relative phenomenon to the timing, self-esteem, an attractiveness level of the person projecting and the person judging.

### The Provider as an Observer/Rater of Attractiveness

Evidence exists that a cosmetic physician's appearance and self-esteem level influence the perceived outcomes for patients seeking esthetic treatments, and there are also reasonable data that physician's workplace satisfaction level may have an impact on patient outcomes. A systemic review evaluating studies of workplace happiness revealed that doctors with better workplace happiness are more likely to be better communicators, offer contagious optimism, and achieve better outcomes in their patients. If doctors have better moods this may manifest in a more positive and upbeat attitude that leads to optimum results perceived by patients. Although the studies evaluating health outcomes and physician interactions are mostly centered in primary care settings, can the same conclusions hold true in esthetics? Satisfaction rates for patients treated with energy-based devices published by dermatology groups have been reported higher than those from plastic surgeons. Outcomes following cosmetic intervention are influenced by multiple variables, included within may be the provider's attitude and their own personal satisfaction with their work.

### Defining Natural

Appearing unnatural is the number one fear hindering potential patients from undergoing a cosmetic medical procedure. What exactly does it mean to appear unnatural, however, is undefined, yet we all can spot an unnatural look. The cosmetic medical community has recently been on a determined quest to define natural, yet it remains frustratingly elusive. It may be that a natural outcome following cosmetic interventions is only but a foggy illusion. Perhaps a better understanding of what is being desired when a patient requests a “natural outcome” would aid in the goal more likely being met. If it can be assumed that to appear better, feel better and not have anyone know treatment was performed, characterizes the correlating outcome signifying “natural,” then a natural outcome is a misnomer. Paradoxically, the patient is really requesting to look unnaturally beautiful yet genuine and feel satisfied. A natural outcome then can only exist as an interpreted deduction. Traditionally, cosmetic medicine has taught and emphasized that to aim for an idealized beauty is a singular mission targeting an incomplete goal that is certain to fall short of patient expectations. Using modern day measures (phi formula, genuineness scales), a person's calculated beauty is at its nadir and intersects with maximum perceived genuineness at a point mathematically defined and suitable to objective scrutiny. This 2-dimensional cross section is then volumized by adding the self-esteem component to get a 3-dimensional cube of attractiveness. It is within an area defined by where the multiple planes are at a maximum that the ever elusive and overly venerated “natural” result can be gleaned as shown in FIG. 2. This area is then subject to variable interpretation based on the judging observer's position from outside the cube. The most ideal outcome for an individual patient requires also considering the relative position of the patient's most meaningful judgers. Therefore, the three-dimensional cube of attractiveness is encased within a four-dimensional space that considers the variable and dynamic positions of others. A treating physician who is also intimately involved in judging the patient's current and desired position would be best to know his or her position in the 4th dimension as well.

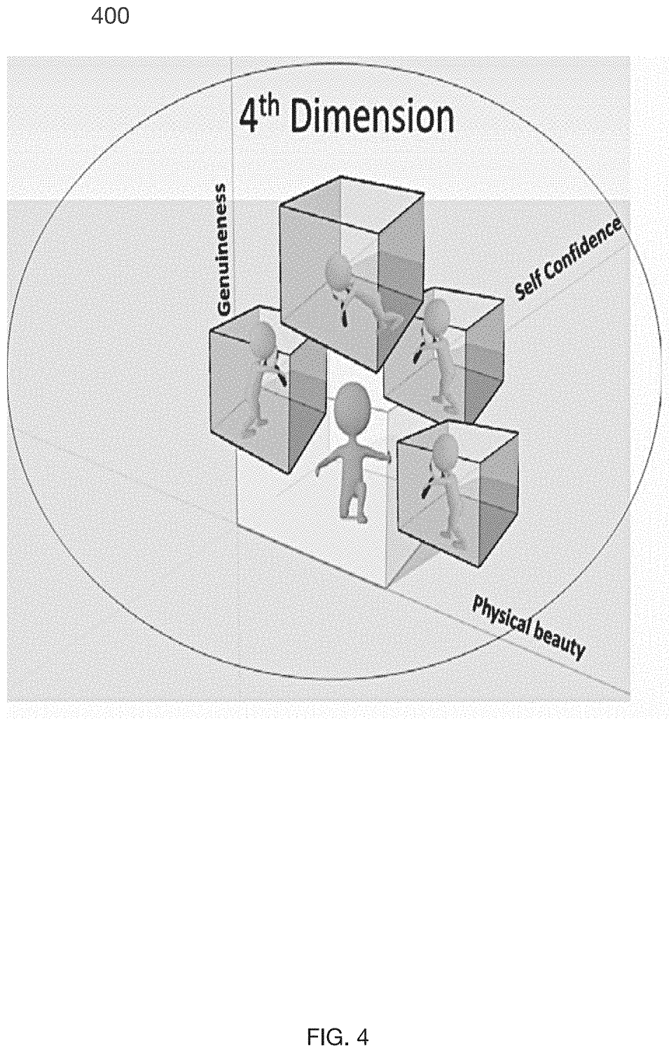

Referring to FIG. 4, several graphs of three dimensional cubes show the difference each type of metric can have on an outcome. To achieve a pleasing natural outcome is only but a relative interpretation of an area within the cube of attractiveness where the three planes of beauty, genuineness, and self-esteem meet. This is then appreciated within a 4th-dimensional plane relative to the judging observer. Systems and method in embodiments for identifying an esthetic ideal of attractiveness are intended to help guide esthetic providers when determining how to best meet their patient's demand for a natural outcome is proposed. The Special Theory of Relativity for Attractiveness creates a mathematically and scientifically logical framework for defining the mission of esthetic medicine. To meet the demands of an individual patient, alleviate the fears of a skeptical populous, and advance the field of cosmetic medicine, the esthetic medical community who serve both as providers and as observers would stand to benefit by understanding the relationship of the four dimensions of attractiveness and how they impact patients, judgers, and themselves.

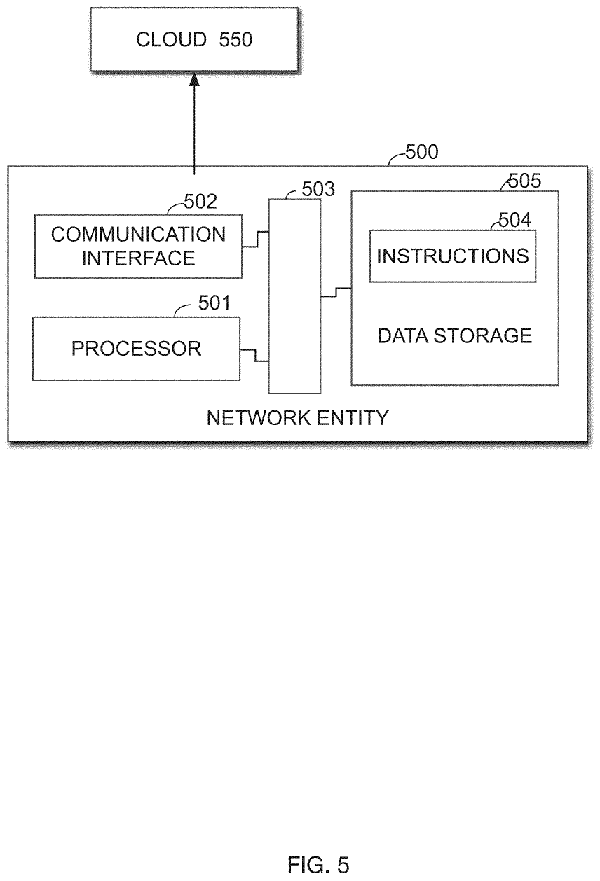

Referring to FIG. 5, a network environment illustrates embodiments in which the different three-dimensional cubes are uses in a computing environment to evaluate and estimate outcomes of cosmetic surgery. More specifically, FIG. 5 illustrates a network entity 500 including a processor or one or more processors 501, a communication interface 502, interoperably coupled to ontology/schema 503. FIG. 5 further shows instructions 504 within data storage 505.

Network entity 500 represents a computing environment capable of collecting sample data associated with cosmetic outcomes, such as from patients or doctors. Network entity 500 can receive input defining schemas in ontology/schema 503 for organizing the collected sample data. Processor 501 profiles the sample data to determine metrics associated with the definitions of beauty described above. Ontology/schema 503 further can include one or more rules engines for developing a functional type system for providing an estimation of cosmetic outcomes.

Some embodiments include generating data according to a procedure-specific schema or data template. Data may be obtained about the parameters of the particular procedure being performed. The schema may be defined using XML or DTD file that specifies the attributes to be collected. For example, a schema specific to liposuction may call for the collection of data pertaining to the location and extent of liposuction being performed, whereas a schema specific to a rhinoplasty may call for the collection of prior and desired nose shape measurements. In addition to procedure-specific parameters, data may be recorded to obtain values for beauty, genuineness and self-esteem. In some embodiments, beauty is calculated using existing formulas such as the phi-formula applied to facial features obtained using pictures of an individual's face. In some embodiments, calculating genuineness includes the measurement of data from observers regarding the perception of an individual. For example, observers may be presented pictures and/or video of an individual via a display device. Video and/or picture data may aim to capture sufficient data of the patient's appearance, e.g. by providing profile, facial views, a plurality of facial expressions, etc. Observers may be prompted to respond to survey questions with regard to their perception of the depicted individual. In some embodiments, responses are captured for each of a plurality of pictures or individual scenes (e.g. a response based solely on a profile view before moving to the next response), whereas in other embodiments, the observer is prompted to rate the observed individual based on an overall impression from the provided imagery. In some embodiments, observers engage in a live viewing of the individual to be observed.

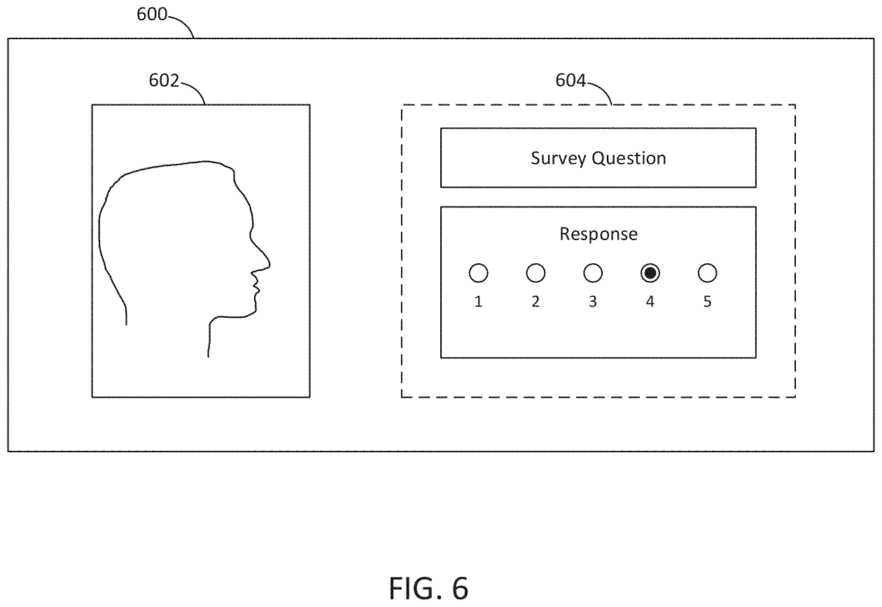

FIG. 6 depicts a display and user interface in accordance with some embodiments. In the example, display 600 is provided by which the observer can view an observed individual and respond to the survey questions. The observed individual is presented in the display at 602. A user interface element 604 displays a survey question and prompts the user for a response. In some embodiments, ratings for genuineness are collected from observers in response to viewing picture(s) and/or video of the observed individual. For example, responses may be gathered by using a 5- or 7-point Likert rating scale. Similarly, a rating for self-esteem may be obtained by collecting responses from the observed individual using a self-esteem scale. In some embodiments, answers to a series of survey questions are collected from observers. The series of survey questions and responses may be used to infer a rating of self-esteem and/or genuineness rather than directly prompting the observer to provide a rating for each metric. In such embodiments, rules may be defined that utilize survey answers to calculate a score for one or more of the metrics of self-esteem and genuineness. For example, survey questions may be weighted differently so that particular responses have lessor or greater effect on the calculated metric.

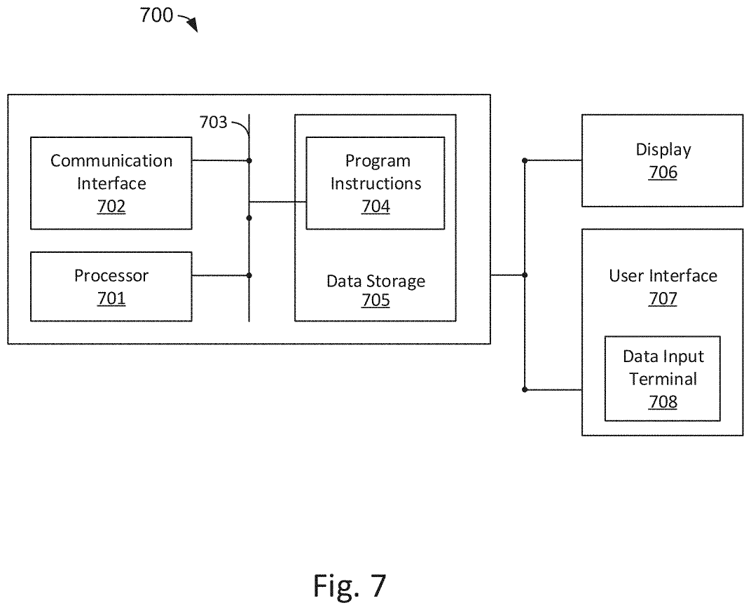

FIG. 7 depicts a system for collecting observer responses. The system 700 includes processor 701, communication interface 702, ontology/schema 703, instructions 704, and data storage 705, each of which may be configured similarly to the corresponding elements of network entity 500 of FIG. 5. The system 700 further includes a display 706 and user interface 707, which may be used for collecting observer responses as described above. The system 700 further includes a data input terminal 708 for receiving observer responses. In some embodiments, the data input terminal is utilized by observers in the manner described above. In some embodiments, one or more sets of observer responses may first be recorded externally to the system 700 (e.g. by paper or electronic survey), and then input via data input terminal 708 (e.g. by a separate party who has collected the responses).

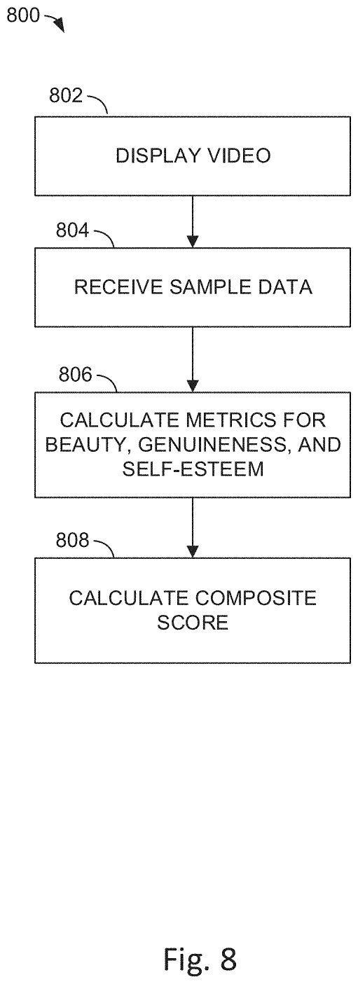

FIG. 8 depicts a method in accordance with some embodiments. The method 800 includes, at 802, displaying video of an individual to be observed. At 804, sample data is received. At 806, metrics for beauty, genuineness, and self-esteem are determined based on the received sample data. At 808, a composite score is calculated. For example, the composite score may represent rated attractiveness based on a volumetric scoring with beauty, genuineness, and self-esteem as the axes, as described above. The composite score may be used as a normalized and/or standardized way of scoring the perceived attractiveness of an individual or, e.g. the perceived naturalness of the outcome of a cosmetic procedure.

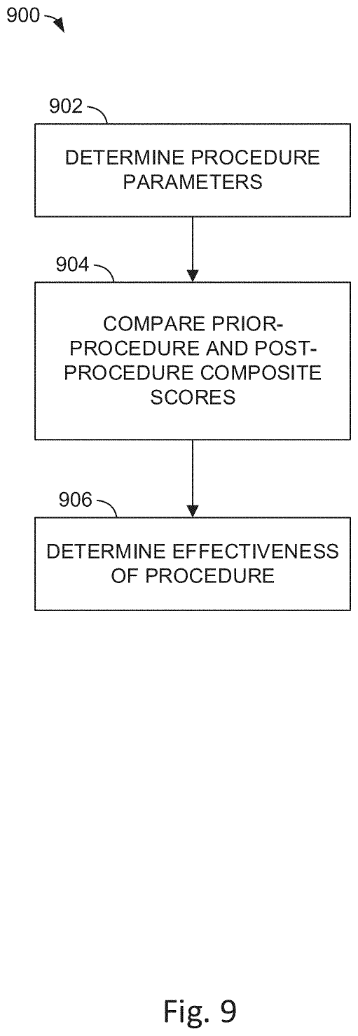

FIG. 9 depicts a second method in accordance with some embodiments. The method 900 includes, at 902, determining the parameters of a procedure. For example, parameters may include a procedure name. In some embodiments, the determining procedure parameters include determining information about the observed individual(s). For example, age, gender, and physical attributes of an individual may be determined. Procedure parameters may be determined at the time of data collection and linked to the metrics and/or composite scores that are collected. At 904, composite scores are determined with respect prior- and post-procedure observations. For example, the data collection methods described above may be performed for one or more individuals both before and after a cosmetic procedure is performed. At 906, the effectiveness of the procedure is determined. For example, a difference between composite scores before and after a procedure may be used as a delta for establishing the success of the cosmetic procedure on an individual. An analysis of a plurality of individuals may be used to establish an effectiveness score. In some embodiments, the effectiveness score is calculated as a difference between pre and post-procedure composite scores. In some embodiments, the effectiveness score is a functional value dependent on the specific before and after score values. This measurement may be used as a normalized and/or standardized way of scoring the outcome of the respective procedure. For example, a cosmetic procedure with a higher effectiveness score may be recommended over a similar or competing cosmetic procedure with a lower effectiveness score. In such examples, it may be helpful to collect data for each of the competing cosmetic procedures using the same schema. In some embodiments, a standardized score calculated in the manner described above is used as a score for cosmetic procedures so that said procedures can be determined to be compliant with a recognized standard. In some embodiments, standardized scores may be utilized to rank procedures for their overall effectiveness. In some embodiments, scores are used as a metric of cosmetic outcome to compare and rank the outcomes of provider of cosmetic procedures (e.g. doctors, clinics, make-up artists, etc.).

In some embodiments, data is collected and linked to pre- and post-procedure observations. In this manner, individuals may be first be rated on their starting composite scores or individual scores of beauty, genuineness, and self-esteem and then be provided a ranking of cosmetic procedures that provide the greatest likelihood of success, e.g. based on their initial scores. Similarly, procedure parameters and data about the individual can be collected and linked to the metrics and composite scores of the observed individuals that are stored in a database. An analysis of this data can provide individuals seeking cosmetic procedures or providers recommending procedures with an indication of procedures that are indicative of providing the best outcomes.

A method for multi-dimensional data integration in a computing environment includes collecting sample data associated with cosmetic outcomes; receiving input defining one or more schemas for organizing the collected sample data; profiling the sample data to determine one or more metrics associated with the sample data, the metrics including at least a physical beauty metric, a genuineness metric and a self-esteem metric; generating one or more rules based on the one or more schemas, the rules including at least an attractiveness rule; and generating a functional estimation system based on the generated one or more rules, for use in processing a data input, the generated functional type system providing an estimation of cosmetic outcome.

In one or more embodiments, the one or more rules includes data rules based on the one or more schemas that are defined in terms of profiled data from a plurality of patients with known cosmetic outcomes; and relationship rules that define associations between patients and one or more judging observers.

In one or more embodiments, the one or more schemas operate as a reference ontology, for use in comparing, classifying, and evaluating a metadata schema for generating three-dimensional volumes defining attractiveness based on the one or more metrics.

In one or more embodiments, the reference ontology provides a variable and dynamic reference; and wherein the collecting sample data associated with cosmetic outcomes includes collecting a judging observer as a metric of cosmetic outcome.

In one or more embodiments, the method is performed in a cloud or cloud-based computing environment.

Another embodiment is directed to a system for ontology analysis of a schema definition for use with a data integration or other computing environment, including one or more processors operable to: collecting sample data associated with cosmetic outcomes; receiving input defining one or more schemas for organizing the collected sample data; profiling the sample data to determine one or more metrics associated with the sample data, the metrics including at least a physical beauty metric, a genuineness metric and a self-esteem metric; generating one or more rules based on the one or more schemas, the rules including at least an attractiveness rule; and generating a functional estimation system based on the generated one or more rules, for use in processing a data input, the generated functional type system providing an estimation of cosmetic outcome.

In one or more embodiments, the one or more rules includes data rules based on the one or more schemas that are defined in terms of profiled data from a plurality of patients with known cosmetic outcomes; and relationship rules that define associations between patients and surgeons.

In one or more embodiments, the one or more schemas operate as a reference ontology, for use in comparing, classifying, and evaluating a metadata schema for generating three-dimensional volumes defining attractiveness based on the one or more metrics. In one or more embodiments, the reference ontology provides a variable and dynamic reference; and wherein the collecting sample data associated with cosmetic outcomes includes collecting a judging observer as a metric of cosmetic outcome.

In one or more embodiments, the system is performed in a cloud or cloud-based computing environment.

Note that various hardware elements of one or more of the described embodiments are referred to as “modules” that carry out (i.e., perform, execute, and the like) various functions that are described herein in connection with the respective modules. As used herein, a module includes hardware (e.g., one or more processors, one or more microprocessors, one or more microcontrollers, one or more microchips, one or more application-specific integrated circuits (ASICs), one or more field programmable gate arrays (FPGAs), one or more memory devices) deemed suitable by those of skill in the relevant art for a given implementation. Each described module may also include instructions executable for carrying out the one or more functions described as being carried out by the respective module, and it is noted that those instructions could take the form of or include hardware (i.e., hardwired) instructions, firmware instructions, software instructions, and/or the like, and may be stored in any suitable non-transitory computer-readable medium or media, such as commonly referred to as RAM, ROM, etc.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable storage media include, but are not limited to, a read only memory (ROM), a random access memory (RAM), a register, cache memory, semiconductor memory devices, magnetic media such as internal hard disks and removable disks, magneto-optical media, and optical media such as CD-ROM disks, and digital versatile disks (DVDs). A processor in association with software may be used to implement a radio frequency transceiver for use in a WTRU, UE, terminal, base station, RNC, or any host computer.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### REFERENCES

1. ASAPS Statistics. www.surgery.org. Accessed Mar. 16, 2018.
2. Swift A, Remington K. Beautiphication a global approach to facial beauty. Clin Plastic Surg. 2011; 38:347-377.
3. Krumhuber E, Can M A. Duchennes Smile be feigned? New evidence on felt and false smiles. Emotions. 2009; 9(6):807-820.
4. Gray-Little B, Williams V, Hancock T D. An item response theory analysis of the Rosenberg Self Esteem scale. Pers Soc Psychol Bull. 1997; 23:443-451.
5. Dayan S H, Arkins J P G. Blinded evaluation of the effects of hyaluronic acid filler on first impression. Dermatol Surg. 2010; 36(Suppl 3):1866-1873.
6. Dayan S H, Lieberman E D, Thakkar N N, Larimer K A, Anstead A. Botulinum toxin a can positively impact first impression. Dematol Surg. 2008; 34(Suppl 1):S40-S47.
7. Dayan S H, Cho K, Siracusa M, Gutierrez-Borst S. Quantifying the impact cosmetic make-up has on age perception and the first impression projected. J Drugs Dermatol. 2015; 14(4):366-374.
8. Freeman J B, Stolier R M, Ingbretsen A Z, Hehman E A. Amygdala responsivity to high-level social information from unseen faces. J Neurosci. 2014; 34(32):10573-10581.
9. Ekman P, Davidson R J, Friesen W V. The Duchenne Smile: emotional expression and brain physiology. II. J Pers Soc Psychol. 1990; 58 (2):342-353.
10. Dayan S H, Arkins J P, Patel A B, Gal T J. A double-blind, randomized, placebo-controlled health-outcomes survey of the effect of botulinum toxin type a injections on quality of life and self-esteem. Der-matol Surg. 2010; 36 (Suppl 4):2088-2097.
11. Baumeister R F, Campbell J D, Krueger J I, Vohs K D. Does high self-esteem cause better performance, interpersonal success, happiness or healthier lifestyles? Psychol Sci Public Interest. 2003; 4:1-44.
12. Harter S. Causes and consequences of low self-esteem in children and adolescents. In: Baumeister R F, ed. Self Esteem: the puzzle of low self-regard. New York: Plenum Press; 1993:87-116.
13. Montoya R M. I'm hot so I'd say you're not: the influence of objective physical attractiveness on mate selection. Pers Soc Psychol Bull. 2008; 34(10):1315-1331.
14. Weingarten G. Pearls before breakfast: can one of the nation's great musicians cut through the fog of a D.C. rush hour? Let's find out. The Washington Post, Apr. 8, 2007.https://www.washingtonpost.com/lifestyle/magazine/pearls-before-breakfast-can-one-of-the-nations-great-musicians-cut-through-the-fog-of-a-dc-rush-hour-lets-find-out/2014/09/23/8a6d46da-4331-11e4-b47c-f5889e061e5f_story.html?utm_term=0.7bb18814d1df. Accessed Mar. 16, 2018.
15. Scheepers R A, Boerebach B C, Arah O A, Heineman M J, Lombarts K M. A systematic review of the impact of physicians' occupational well-being on the quality of patients care. Int J Behav Med. 2015; 22 (6):683-698.
16. Tavel M E. The placebo effect: the good, the bad, and the ugly. Am J Med. 2014; 127(6):484-488.
17. Rousseaux I, Robson S. Body contouring and skin tightening using a unique novel multisource radiofrequency energy delivery method. J Clin Aesthet Dermatol. 2017; 10(4):24-29.
18. Georgette O, Ron H, Sumeet T, Spencer B, Kenkel J M. Evaluation of a microfocused ultrasound system for improving skin laxity and tightening in the lower face. Aesthet Surg J. 2014; 34(7):1099-1110.
19. Medscape Physician compensation Report, 2015. https://www.medscape.com/sites/public/physician-comp/2015. Accessed May 14, 2018.
20. AAFPRS Doctor Survey. https://www.prnewswire.com/news-re-leases/aafprs-annual-survey-reveals-trends-in-facial-plastic-surgery-300588707.html. Accessed Jun. 12, 2018.

What is claimed:

1. A method for multi-dimensional data integration for estimating cosmetic outcomes in a computing environment comprising:
receiving input defining one or more schemas for organizing cosmetic procedure data;
generating visual depictions of subjects related to a predetermined cosmetic procedure and obtaining one or more metrics associated with the predetermined cosmetic procedure, the metrics including at least a physical beauty metric, a genuineness metric and a self-esteem metric;
obtaining one or more rules based on the one or more schemas, the rules including at least an attractiveness rule; and
generating a functional estimation system based on the generated one or more rules to calculate a composite attractiveness score, for use in processing the one or more metrics, the generated functional system providing an estimation of cosmetic outcome.

2. The method of claim 1, further comprising measuring one or more metrics associated with visual depictions of the subject prior to undergoing the predetermined cosmetic procedure, the metrics including at least a physical beauty metric, a genuineness metric and a self-esteem metric.

3. The method of claim 1 wherein the beauty metric is measured using a phi formula, the genuineness metric is measured by a Likert scale, and the self-esteem metric is measured by a self-rated self-esteem scale.

4. The method of claim 3 wherein the cosmetic procedure data associated with cosmetic outcomes includes collecting a judging observer as a metric of cosmetic outcome.

5. The method of claim 1 wherein the method is performed in a cloud or cloud-based computing environment.

6. The method of claim 1 wherein the functional estimation system includes a user interface for interacting with a user to enable determining an estimation of outcome of cosmetic intervention including one or more of makeup, hair care, neurotoxins, fillers, surgery and hormonal replacement.

7. The method of claim 1 wherein the functional estimation system comprises an estimation system for determining an estimation of outcome of cosmetic intervention including one or more of makeup, hair care, neurotoxins, fillers, surgery and hormonal replacement.

8. A system for ontology analysis of a schema definition for use with a data integration or other computing environment, comprising:
one or more processors operable to:
receive input defining one or more schemas for organizing cosmetic procedure data;
generate visual depictions of subjects related to a predetermined cosmetic procedure and obtaining one or more metrics associated with the sample data, the metrics including at least a physical beauty metric, a genuineness metric and a self-esteem metric;
obtain one or more rules based on the one or more schemas, the rules including at least an attractiveness rule; and
generate a functional estimation system based on the generated one or more rules, to calculate a composite attractiveness score by processing the one or more metrics, the generated functional type system providing an estimation of cosmetic outcome.

9. The system of claim 8, wherein the one or more rules includes data rules based on the one or more schemas that are defined in terms of profiled data from a plurality of patients with known cosmetic outcomes; and
relationship rules that define associations between patients and surgeons.

10. The system of claim 8, wherein the beauty metric is measured using a phi formula, the genuineness metric is measured by a Likert scale, and the self-esteem metric is measured by a self-rated self-esteem scale.

11. The system of claim 10 wherein the cosmetic procedure data associated with cosmetic outcomes includes collecting a judging observer as a metric of cosmetic outcome.

12. The system of claim 8, wherein the system is performed in a cloud or cloud-based computing environment.

13. The system of claim 8 wherein the functional estimation system includes a user interface for interacting with a user to enable determining an estimation of outcome of cosmetic intervention including one or more of makeup, hair care, neurotoxins, fillers, surgery and hormonal replacement.

14. The system of claim 8 wherein the functional estimation system comprises an estimation system for determining an estimation of outcome of cosmetic intervention including one or more of makeup, hair care, neurotoxins, fillers, surgery and hormonal replacement.

15. A non-transitory computer readable storage medium, including instructions stored thereon which when read and executed by one or more computers cause the one or more computers to perform a method comprising:
receiving input defining one or more schemas for organizing cosmetic procedure data;
generating visual depictions of subjects related to a predetermined cosmetic procedure and obtaining one or more metrics associated with the cosmetic procedure data, the metrics including at least a physical beauty metric, a genuineness metric and a self-esteem metric; generating one or more rules based on the one or more schemas, the rules including at least an attractiveness rule; and
generating a functional estimation system based on the generated one or more rules, for use in processing a data input, the generated functional type system providing an estimation of cosmetic outcome.

16. The non-transitory computer readable storage medium of claim 13 wherein the functional estimation system includes a user interface for interacting with a user to enable determining an estimation of outcome of cosmetic intervention including one or more of makeup, hair care, neurotoxins, fillers, surgery and hormonal replacement.
